SEQUENCE LISTING (30 sequences)

SEQ ID NO: 1 - protein, 161 aa, Artificial Sequence
  Amino acid sequence of 1A1
GSDLGKKLLEAARAGRDDEVRILMANGADVNAGDASGYTPLHLAAYNGHLEIVEVLLKHGADVNAEDLLGMTPLHLAAPFGHLEIVEVLLKHGADVNAKNRNNGKTPLHLAAFVGHLEIVEVLLKNGADVNAQDKFGKTAFDISIDNGNEDLAEILQKLNG

SEQ ID NO: 2 - DNA, 485 bp, Artificial Sequence
  Nucleotide sequence of 1A1
ggcagcgatctgggcaaaaaactgctggaagccgcccgcgccggccgcgatgatgaagttcgcatcctgatggccaacggtgcggatgttaacgcgggtgatgcttcgggttatacccctctgcatctggccgcctataatgggcatctggaaatcgttgaggttctgctgaaacatggcgcagatgttaatgccgaggatctgctgggtatgacgccactgcatctggcggccccttttggccacctcgaaatagttgaggttcttttgaagcatggcgccgacgtaaacgccaagaatcggaataatggtaagactccgttgcacctggccgcatttgttgggcacctggagatcgtggaggttctcctcaaaaatggcgccgatgtcaatgcacaagataaattcggcaaaaccgccttcgatatcagcatcgataatggcaatgaagatctggccgaaatcctgcagaaactgaatggcca

SEQ ID NO: 3 - protein, 393 aa, Artificial Sequence
  1A1-Fc recombinant protein
GSDLGKKLLEAARAGRDDEVRILMANGADVNAGDASGYTPLHLAAYNGHLEIVEVLLKHGADVNAEDLLGMTPLHLAAPFGHLEIVEVLLKHGADVNAKNRNNGKTPLHLAAFVGHLEIVEVLLKNGADVNAQDKFGKTAFDISIDNGNEDLAEILQKLNGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 232 aa, Artificial Sequence
  Amino acid sequence of Fc
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 122 aa, Artificial Sequence
  Heavy chain of Tezepelumab
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 6 - protein, 108 aa, Artificial Sequence
  Light chain of Tezepelumab
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 7 - protein, 161 aa, Artificial Sequence
  Amino acid sequence of G7W1
GSDLGKKLLEAAWAGRDDEVRILMANGADVNAGDASGDTPLHLAAVVGHLEIVEVLLKHGADVNAEDLLGMTPLHLAAPFGHLEIVEVLLKHGADVNAAARNNGKTPLHLAAFVGHLEIVEVLLKNGADVNAQDKFGKTAFDISIDNGNEDLAEILQKLNG

SEQ ID NO: 8 - protein, 163 aa, Artificial Sequence
  Amino acid sequence of 3H10
GSHMDLGKKLLEAAWAGRDDEVRILMANGADVNAGDASGDTPLHLAAFSGHLEIVEVLLKHGADVNAEDLLGMTPLHLAAPFGHLEIVEVLLKHGADVNAAARNNGKTPLHLAAFVGHLEIVEVLLKNGADVNAQDKFGKTAFDISIDNGNEDLAEILQKLNG

SEQ ID NO: 9 - DNA, 489 bp, Artificial Sequence
  Nucleotide sequence of 3H10
ggcagccatatggatctgggcaaaaaactgctggaagcagcctgggcaggtcgtgatgatgaagtgcgtattctgatggccaatggtgcagatgtgaatgccggtgacgccagcggtgacaccccgttacatctggcagcctttagcggccatctggaaattgtggaagttctgctgaaacatggtgccgatgtgaatgcggaagatctgctgggcatgaccccgctgcatctggcagcaccgtttggtcatctggaaatcgttgaagtgctgctgaaacacggtgccgatgttaatgccgccgcccgtaataatggtaaaaccccgctgcacctggccgcatttgttggtcatctggagattgtggaagtgctgttaaaaaatggcgccgatgtgaacgcacaggataaatttggcaaaaccgcatttgatattagtattgataacggtaacgaggatctggcagaaattctgcagaaactgaatggt

SEQ ID NO: 10 - protein, 4 aa, Artificial Sequence
  The first binding active site of the first binding domain
GDAS

SEQ ID NO: 11 - protein, 4 aa, Artificial Sequence
  The first binding active site of the second binding domain
EDLL

SEQ ID NO: 12 - protein, 5 aa, Artificial Sequence
  The first binding active site of the third binding domain
KNRNN

SEQ ID NO: 13 - protein, 5 aa, Artificial Sequence
  The first binding active site of the third binding domain
AARNN

SEQ ID NO: 14 - protein, 7 aa, Artificial Sequence
  The second framework site
TPLHLAA

SEQ ID NO: 15 - protein, 11 aa, Artificial Sequence
  The third framework site
GHLEIVEVLLK

SEQ ID NO: 16 - protein, 6 aa, Artificial Sequence
  The fourth framework site
GADVNA

SEQ ID NO: 17 - protein, 131 aa, Artificial Sequence
  Human TSLP
YDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKARKAKVTTNKCLEQVSQLQGLWRRFNRPLLKQQ

SEQ ID NO: 18 - protein, 32 aa, Artificial Sequence
  N-terminal Capping region
GSDLGKKLLEAARAGRDDEVRILMANGADVNA

SEQ ID NO: 19 - protein, 32 aa, Artificial Sequence
  N-terminal Capping region
GSDLGKKLLEAAWAGRDDEVRILMANGADVNA

SEQ ID NO: 20 - protein, 34 aa, Artificial Sequence
  N-terminal Capping region
GSHMDLGKKLLEAAWAGRDDEVRILMANGADVNA

SEQ ID NO: 21 - protein, 29 aa, Artificial Sequence
  C-terminal Capping region
QDKFGKTAFDISIDNGNEDLAEILQKLNG

SEQ ID NO: 22 - protein, 34 aa, Artificial Sequence
  Binding domain
  SITE (1)..(1): X is G, E, K or A
  SITE (2)..(2): X is D, N or A
  SITE (3)..(3): X is A, L or R
  SITE (4)..(4): X is S, L or N
  SITE (5)..(5): X is N or absent
  SITE (7)..(7): X is D, Y, M or K
  SITE (15)..(15): X is V, Y, P or F
  SITE (16)..(16): X is V, N or F
  SITE (28)..(28): X is H, N or K
XXXXXGXTPLHLAAXXGHLEIVEVLLKXGADVNA

SEQ ID NO: 23 - protein, 33 aa, Artificial Sequence
  Binding domain
GDASGYTPLHLAAYNGHLEIVEVLLKHGADVNA

SEQ ID NO: 24 - protein, 33 aa, Artificial Sequence
  Binding domain
EDLLGMTPLHLAAPFGHLEIVEVLLKHGADVNA

SEQ ID NO: 25 - protein, 34 aa, Artificial Sequence
  Binding domain
KNRNNGKTPLHLAAFVGHLEIVEVLLKNGADVNA

SEQ ID NO: 26 - protein, 33 aa, Artificial Sequence
  Binding domain
GDASGDTPLHLAAVVGHLEIVEVLLKHGADVNA

SEQ ID NO: 27 - protein, 33 aa, Artificial Sequence
  Binding domain
GDASGDTPLHLAAFSGHLEIVEVLLKHGADVNA

SEQ ID NO: 28 - protein, 34 aa, Artificial Sequence
  Binding domain
AARNNGKTPLHLAAFVGHLEIVEVLLKNGADVNA

SEQ ID NO: 29 - protein, 34 aa, Artificial Sequence
  N-terminal Capping region
  SITE (3)..(3): X is H or absent
  SITE (4)..(4): X is M or absent
  SITE (15)..(15): XRW
  SITE (15)..(15): X is R or W
GSXXDLGKKLLEAAXAGRDDEVRILMANGADVNA

SEQ ID NO: 30 - DNA, 480 bp, Artificial Sequence
  Nucleotide sequence of G7W1
ggcagcgatctgggcaaaaaactgctggaagccgcctgggccggccgcgatgatgaagttcgcatcctgatggccaacggtgcggatgttaacgcgggtgatgcttcgggtgatacccctctgcatctggccgccgttgttgggcatctggaaatcgttgaggttctgctgaaacatggcgcagatgttaatgccgaggatctgctgggtatgacgccactgcatctggcggccccttttggccacctcgaaatagttgaggttcttttgaagcatggcgccgacgtaaacgccgctgctcggaataatggtaagactccgttgcacctggccgcatttgttgggcacctggagatcgtggaggttctcctcaaaaatggcgccgatgtcaatgcacaagataaattcggcaaaaccgccttcgatatcagcatcgataatggcaatgaagatctggccgaaatcctgcagaaactgaat

(19) United States
(12) Patent Application Publication
(10) Pub. No.: US 2025/0376501 A1
(43) Pub. Date: Dec. 11, 2025

(54) RECOMBINANT BINDING PROTEIN TARGETING TSLP AND USE THEREOF

(71) Applicant: JINGYUAN BIOSCIENCES (SUZHOU) CO., LTD., Jiangsu (CN)

(72) Inventors: Xiaobo CHEN, Jiangsu (CN); Linghua GUO, Jiangsu (CN); Yunqiu YAN, Jiangsu (CN); Wenzheng QIAN, Jiangsu (CN); Hui CHAI, Jiangsu (CN); Minxiang LIU, Jiangsu (CN)

(21) Appl. No.: 18/876,668

(22) PCT Filed: Jun. 29, 2022
(86) PCT No.: PCT/CN2022/102471

§ 371 Date: Dec. 19, 2024

(51) Int. Cl. C07K 14/705; A61K 38/00

(52) U.S. Cl. CPC C07K 14/705; A61K 38/00; C07K 2319/30

(57) ABSTRACT

Disclosed are a recombinant binding protein targeting TSLP and the use thereof. The recombinant binding protein comprises at least one ankyrin repeat domain that specifically binds to TSLP, wherein the ankyrin repeat domain comprises three tandem binding domains, and each binding domain comprises four binding active regions. The recombinant binding protein of the present invention has excellent binding activity to TSLP, and effectively blocks the interaction between TSLP and a receptor thereof, thereby effectively controlling TSLP-related diseases.

### TECHNICAL FIELD

The present disclosure belongs to the field of biomedicine, and specifically relates to a recombinant binding protein targeting TSLP and a use thereof.

### BACKGROUND

Thymic stromal lymphopoietin (TSLP), a cytokine of the interleukin-7 family, is mainly produced by epithelial cells and keratinocytes located in the skin, intestinal tract, or lung, and promote allergic inflammatory responses by activating dendritic cells (DCs), mast cells, and eosinophils, resulting in the generation of a large amount of allergic cytokines. Currently, studies have shown that TSLP plays an important role in the following underlying allergic diseases, such as atopic disease (atopic dermatitis, allergic rhinitis, asthma), eosinophilic esophagitis, and chronic obstructive pulmonary disease (COPD), etc. In terms of patients with atopic dermatitis, a large amount of TSLP can be detected in the damaged skin and few in the undamaged skin. Meanwhile, overexpression of TSLP can be detected in submucosal cells of patients with COPD and in lung epithelial cells of patients with asthma.

Human TSLP cDNA encodes a precursor protein of 159 amino acid (aa) residues and has a signal sequence of 28 amino acids. The TSLP molecule contains three pairs of disulfide bonds and two N-glycosylation sites, with a molecular weight of approximately 18 to 21 kDa. TSLP cytokines exert their biological functions mainly through TSLPR/IL-7Rα receptors on the surface of various immune cells. The IL-7Rα chain is expressed in immune cells, but the other chain, TSLPR, is only expressed in dendritic cells. Typically, TSLP transduces signals through the JAK/STAT (JAK kinase-signal transducer and activator of transcription) pathway. Firstly, TSLP weakly binds to the TSLPR receptor, and then the complex binds to IL-7Rα with high affinity to form a stable TSLP-TSLPR-IL-7Rα receptor complex. The intracellular segment of TSLPR receptor recruits and activates JAK2, and co-functions with JAK1 recruited by the intracellular segment of IL-7Rα receptor to activate multiple downstream STAT signaling molecules, thereby inducing the activation of DC cells during the induction stage of the immune response, which is critical to promoting the differentiation of helper T cells (T helper 2 cells, TH2 cells) and secretion of TH2 factors. Meanwhile, TSLP can induce the proliferation of mast cells, prolong the survival period of eosinophils, and promote the specific release of inflammatory cytokines and chemokines.

Therefore, inhibiting the formation of the complex between TSLP and TSLPR/IL-7Rα is advantageous in achieving the purpose of intervening inflammation at early stage, preventing immune cells such as dendritic cells from releasing pro-inflammatory cytokines.

DARPin (Designated Ankyrin Repeat Protein) refers to a non-antibody protein with high specificity and high binding affinity for a target protein, consisting of closely packed ankyrin repeat sequences. DARPin originates from a natural ankyrin and is typically formed from two or more binding motifs contained between N-terminal and C-terminal motifs (commonly referred to as N-terminal or C-terminal “cap”) that shield a hydrophobic region. Multiple binding motifs in tandem form a large domain and thus mediate protein-protein interactions.

Currently, biomolecules targeting TSLP are antibodies. However, in addition to antibodies, the prior art lacks a novel binding protein or binding domain that can be used to specifically bind to the target molecule and thus act as an antagonist, such as biomolecules designed as ankyrin repeat proteins or domains, thereby providing more options for new drug development and patient medication.

### SUMMARY

The technical problem to be addressed by the present disclosure is to overcome the defect in the prior art of lacking an ankyrin repeat protein targeting TSLP or a protein comprising an ankyrin repeat domain. The present disclosure provides a recombinant binding protein targeting TSLP and a use thereof. The recombinant binding protein of the present disclosure can bind to TSLP, block the formation of a complex between TSLP and the receptor TSLPR/IL-7Rα, inhibit pro-inflammatory signaling that promotes type II immune response, thereby effectively controlling TSLP-related diseases such as allergic diseases.

The present disclosure addresses the above technical problem by the following technical solutions.

A first aspect of the present disclosure provides a recombinant binding protein targeting TSLP; the recombinant binding protein comprises at least one ankyrin repeat domain that specifically binds to TSLP;

the ankyrin repeat domain comprises three tandem binding domains; the amino acid sequence of the binding domain is as shown in SEQ ID NO: 22.

In the present disclosure, the amino acid sequence as shown in SEQ ID NO: 22 is X₁X₂X₃X₄X₅GX₆TPLHLAAX₇X₈GHLEIVEVLLKX₉GADVNA, wherein X₁ is G, E, K, or A; X₂ is D, N, or A; X₃ is A, L, or R; X₄ is S, L, or N; X₅ is N or absent; X₆ is D, Y, M, or K; X₇ is V, Y, P, or F; X₈ is V, N, or F; X₉ is H, N, or K.

In some embodiments of the present disclosure, the amino acid sequence of the binding domain is as shown in any one of SEQ ID NOs: 23 to 28.

In the present disclosure, the three tandem binding domains are a first binding domain, a second binding domain, and a third binding domain, respectively; wherein the amino acid sequence of the first binding domain is as shown in SEQ ID NO: 23, SEQ ID NO: 26, or SEQ ID NO: 27; the amino acid sequence of the second binding domain is as shown in SEQ ID NO: 24; the amino acid sequence of the third binding domain is as shown in SEQ ID NO: 25 or SEQ ID NO: 28.

In some embodiments of the present disclosure, the ankyrin repeat domain further comprises an N-terminal capping region and a C-terminal capping region; the amino acid sequence of the N-terminal capping region is as shown in SEQ ID NO: 29; the amino acid sequence of the C-terminal capping region is as shown in SEQ ID NO: 21.

In the present disclosure, the amino acid sequence as shown in SEQ ID NO: 29 is GSX₁₀X₁₁DLGKKLLEAAX₁₂AGRDDEVRILMANGADVNA, wherein X₁₀ is H or absent; X₁₁ is M or absent; X₁₂ is R or W.

In some specific embodiments of the present disclosure, the amino acid sequence of the N-terminal capping region is as shown in any one of SEQ ID NOs: 18 to 20.

In the present disclosure, the amino acid sequence as shown in SEQ ID NO: 18 is GSDLGKKLLEAARAGRDDEVRILMANGADVNA; the amino acid sequence as shown in SEQ ID NO: 19 is GSDLGKKLLEAAWAGRDDEVRILMANGADVNA; the amino acid sequence as shown in SEQ ID NO: 20 is GSHMDLGKKLLEAAWAGRDDEVRILMANGADVNA; the amino acid sequence as shown in SEQ ID NO: 21 is QDKFGKTAFDISIDNGNEDLAEILQKLNG; the amino acid sequence as shown in SEQ ID NO: 23 is GDASGYTPLHLAAYNGHLEIVEVLLKHGADVNA; the amino acid sequence as shown in SEQ ID NO: 24 is EDLLGMTPLHLAAPFGHLEIVEVLLKHGADVNA; the amino acid sequence as shown in SEQ ID NO: 25 is KNRNNGKTPLHLAAFVGHLEIVEVLLKNGADVNA; the amino acid sequence as shown in SEQ ID NO: 26 is GDASGDTPLHLAAVVGHLEIVEVLLKHGADVNA; the amino acid sequence as shown in SEQ ID NO: 27 is GDASGDTPLHLAAFSGHLEIVEVLLKHGADVNA; the amino acid sequence as shown in SEQ ID NO: 28 is AARNNGKTPLHLAAFVGHLEIVEVLLKNGADVNA.

A second aspect of the present disclosure provides a recombinant binding protein targeting TSLP; the recombinant binding protein comprises at least one ankyrin repeat domain that specifically binds to TSLP; the ankyrin repeat domain comprises three tandem binding domains, and each binding domain comprises four binding active sites;
wherein the three binding domains are a first binding domain, a second binding domain, and a third binding domain in tandem; the first binding domain, the second binding domain, the third binding domain, and a fourth binding domain each comprise a first binding active site, a second binding active site, a third binding active site, and a fourth binding active site in tandem;
the amino acid sequence of the first binding active site of the first binding domain is as shown in SEQ ID NO: 10, the amino acid residue of the second binding active site is Y or D, the amino acid sequence of the third binding active site is YN, VV, or FS, and the amino acid residue of the fourth binding active site is H;
the amino acid sequence of the first binding active site of the second binding domain is as shown in SEQ ID NO: 11, the amino acid residue of the second binding active site is M, the amino acid sequence of the third binding active site is PF, and the amino acid residue of the fourth binding active site is H;
the amino acid sequence of the first binding active site of the third binding domain is as shown in SEQ ID NO: 12 or SEQ ID NO: 13, the amino acid residue of the second binding active site is K, the amino acid sequence of the third binding active site is FV, and the amino acid residue of the fourth binding active site is N.

In some embodiments of the present disclosure, the amino acid sequence of the first binding active site of the first binding domain is as shown in SEQ ID NO: 10, the amino acid residue of the second binding active site is Y, the amino acid sequence of the third binding active site is YN, and the amino acid residue of the fourth binding active site is H; the amino acid sequence of the first binding active site of the second binding domain is as shown in SEQ ID NO: 11, the amino acid residue of the second binding active site is M, the amino acid sequence of the third binding active site is PF, and the amino acid residue of the fourth binding active site is H; the amino acid sequence of the first binding active site of the third binding domain is as shown in SEQ ID NO: 12, the amino acid residue of the second binding active site is K, the amino acid sequence of the third binding active site is FV, and the amino acid residue of the fourth binding active site is N.

In some embodiments of the present disclosure, the amino acid sequence of the first binding active site of the first binding domain is as shown in SEQ ID NO: 10, the amino acid residue of the second binding active site is D, the amino acid sequence of the third binding active site is VV, and the amino acid residue of the fourth binding active site is H; the amino acid sequence of the first binding active site of the second binding domain is as shown in SEQ ID NO: 11, the amino acid residue of the second binding active site is M, the amino acid sequence of the third binding active site is PF, and the amino acid residue of the fourth binding active site is H; the amino acid sequence of the first binding active site of the third binding domain is as shown in SEQ ID NO: 13, the amino acid residue of the second binding active site is K, the amino acid sequence of the third binding active site is FV, and the amino acid residue of the fourth binding active site is N.

In some embodiments of the present disclosure, the amino acid sequence of the first binding active site of the first binding domain is as shown in SEQ ID NO: 10, the amino acid residue of the second binding active site is D, the amino acid sequence of the third binding active site is FS, and the amino acid residue of the fourth binding active site is H; the amino acid sequence of the first binding active site of the second binding domain is as shown in SEQ ID NO: 11, the amino acid residue of the second binding active site is M, the amino acid sequence of the third binding active site is PF, and the amino acid residue of the fourth binding active site is H; the amino acid sequence of the first binding active site of the third binding domain is as shown in SEQ ID NO: 13, the amino acid residue of the second binding active site is K, the amino acid sequence of the third binding active site is FV, and the amino acid residue of the fourth binding active site is N.

In the present disclosure, the binding domain further comprises a framework site; the framework site comprises a first framework site, a second framework site, a third framework site, and a fourth framework site sequentially; the binding activity site and the framework site are arranged with intervals; wherein the amino acid residue of the first framework site is G, the amino acid sequence of the second framework site is as shown in SEQ ID NO: 14, the amino acid sequence of the third framework site is as shown in SEQ ID NO: 15, and the amino acid sequence of the fourth framework site is shown as SEQ ID NO: 16.

In the present disclosure, the framework site is a conserved site of the binding domain. It is known to those skilled in the art that the conserved site of the binding domain has low variation activity in the ankyrin repeat protein, i.e., the conserved sites of different ankyrin repeat proteins with the same number of repeat fragments for different targets or the same target are almost identical.

In the present disclosure, the amino acid sequence of SEQ ID NO: 10 is GDAS; the amino acid sequence of SEQ ID NO: 11 is LL; the amino acid sequence of SEQ ID NO: 12 is KNRNN; the amino acid sequence of SEQ ID NO: 13 is AARNN; the amino acid sequence of SEQ ID NO: 14 is TPLHLAA; the amino acid sequence of SEQ ID NO: 15 is GHLEIVEVLLK; the amino acid sequence of SEQ ID NO: 16 is GADVNA.

In some embodiments of the present disclosure, the N-terminus and C-terminus of the ankyrin repeat domain further comprise a capping sequence; the capping sequence at the N-terminus and the capping sequence at the C-terminus are as described in the N-terminal capping region and the C-terminal capping region of the first aspect.

In the present disclosure, at least one ankyrin repeat domain of the recombinant binding protein has the amino acid sequence as shown in SEQ ID NO: 1, 7, or 8.

In some specific embodiments of the present disclosure, the nucleotide sequence encoding the amino acid sequence as shown in SEQ ID NO: 1 is as shown in SEQ ID NO: 2.

In some specific embodiments of the present disclosure, the nucleotide sequence encoding the amino acid sequence as shown in SEQ ID NO: 7 is as shown in SEQ ID NO: 30.

In some specific embodiments of the present disclosure, the nucleotide sequence encoding the amino acid sequence as shown in SEQ ID NO: 8 is as shown in SEQ ID NO: 9.

In the present disclosure, the recombinant binding protein comprises two, three, or four ankyrin repeat domains.

In some embodiments of the present disclosure, at least one ankyrin repeat domain of the recombinant binding protein has an amino acid sequence that is at least 98%, preferably at least 99% identical to the amino acid sequence as shown in SEQ ID NO: 1, 7, or 8.

A third aspect of the present disclosure provides a fusion protein targeting TSLP, wherein the fusion protein comprises a structurally stable protein and the recombinant binding protein according to the first aspect; the structurally stable protein is used for prolonging the in vivo plasma half-life of the recombinant binding protein.

In the present disclosure, the structurally stable protein may be conventional in the art, preferably selected from an anti-HSA protein, an HSA protein, and an antibody Fc fragment.

In some embodiments of the present disclosure, the structurally stable protein is an antibody Fc fragment, and the amino acid sequence of the antibody Fc fragment is preferably as shown in SEQ ID NO: 4.

A fourth aspect of the present disclosure provides an isolated nucleic acid encoding the recombinant binding protein according to the first aspect, the recombinant binding protein according to the second aspect, or the fusion protein according to the third aspect.

A fifth aspect of the present disclosure provides a recombinant expression vector comprising the nucleic acid according to the fourth aspect.

In the present disclosure, the recombinant expression vector may be conventional in the art, and is preferably a plasmid, cosmid, phage, or viral vector.

In some embodiments of the present disclosure, the viral vector is a retroviral vector, a lentiviral vector, an adenoviral vector, or an adeno-associated viral vector.

A sixth aspect of the present disclosure provides a transformant, wherein the transformant has a host cell comprising the recombinant expression vector according to the fifth aspect.

In some embodiments of the present disclosure, the host cell is a prokaryotic or eukaryotic cell.

In some embodiments of the present disclosure, the host cell is a mammalian cell.

A seventh aspect of the present disclosure provides a recombinant cell comprising the recombinant binding protein according to the first aspect, the recombinant binding protein according to the second aspect, or the fusion protein according to the third aspect.

In some embodiments of the present disclosure, the recombinant cell is derived from a mammalian cell line or a human cell line.

In some embodiments of the present disclosure, the recombinant cell is derived from a mammalian cell line, such as a CHO cell.

An eighth aspect of the present disclosure provides a method for preparing a recombinant binding protein targeting TSLP or a fusion protein targeting TSLP, comprising culturing the transformant according to the sixth aspect, and obtaining the recombinant binding protein targeting TSLP or the fusion protein targeting TSLP from the culture of the transformant.

A ninth aspect of the present disclosure provides a pharmaceutical composition comprising the recombinant binding protein according to the first aspect, the recombinant binding protein according to the second aspect, or the fusion protein according to the third aspect, and a pharmaceutically acceptable carrier.

In the present disclosure, the pharmaceutically acceptable carrier may be a carrier commonly used in protein preparations, preferably one or more selected from lactose, glucose, sucrose, sorbitol, mannitol, starch, arabic gum, calcium phosphate, alginate, gelatin, calcium silicate, microcrystalline cellulose, polyvinylpyrrolidone, water, syrup, methylcellulose, methyl hydroxybenzoate, propyl hydroxybenzoate, talc, magnesium stearate, and mineral oil.

In some embodiments of the present disclosure, the pharmaceutical composition further comprises an excipient, wherein the excipient is preferably one or more selected from a lubricant, a wetting agent, a sweetening agent, a flavoring agent, an emulsifier, and a suspending agent.

A tenth aspect of the present disclosure provides a use of the recombinant binding protein according to the first aspect in the manufacture of a medicament for diagnosing, preventing, and/or treating an autoimmune disease, an inflammatory disease, and/or an allergic disease.

An eleventh aspect of the present disclosure provides a use of the recombinant binding protein according to the second aspect in the manufacture of a medicament for diagnosing, preventing, and/or treating an autoimmune disease, an inflammatory disease, and/or an allergic disease.

A twelfth aspect of the present disclosure provides a use of the fusion protein according to the third aspect in the manufacture of a medicament for diagnosing, preventing and/or treating an autoimmune disease, an inflammatory disease, and/or an allergic disease.

A thirteenth aspect of the present disclosure provides a use of the recombinant cell according to the seventh aspect in the manufacture of a medicament for diagnosing, preventing and/or treating an autoimmune disease, an inflammatory disease, and/or an allergic disease.

A fourteenth aspect of the present disclosure provides a use of the pharmaceutical composition according to the ninth aspect in the manufacture of a medicament for diagnosing, preventing and/or treating an autoimmune disease, an inflammatory disease, and/or an allergic disease.

In some embodiments of the present disclosure, the autoimmune disease is selected from rheumatoid arthritis and multiple sclerosis.

In some embodiments of the present disclosure, the inflammatory disease is selected from ulcerative colitis, eosinophilic esophagitis, chronic obstructive pulmonary disease, and psoriasis.

In some embodiments of the present disclosure, the allergic disease is selected from atopic dermatitis, allergic rhinitis, allergic conjunctivitis, asthma, and allergic sinusitis.

A fifteenth aspect of the present disclosure provides a drug box kit comprising a drug box A and a drug box B;
the drug box A comprises the recombinant binding protein according to the first aspect, the recombinant binding protein according to the second aspect, the fusion protein according to the third aspect, the recombinant cell according to the seventh aspect, and/or the pharmaceutical composition according to the ninth aspect; the drug box B comprises other therapeutic agents.

In some embodiments of the present disclosure, the drug box A and the drug box B are administered simultaneously or sequentially.

In the present disclosure, the administration may be oral administration or parenteral administration. The parenteral administration may be a conventional route of administration in the art, preferably intravenous, subcutaneous, intramuscular, intraperitoneal, endothelial, topical, intranasal, intrapulmonary, or rectal administration.

A sixteenth aspect of the present disclosure provides a method for preventing and/or treating an autoimmune disease, an inflammatory disease, and/or an allergic disease, comprising administering to a patient in need thereof an effective amount of the recombinant binding protein according to the first aspect, the recombinant binding protein according to the second aspect, the fusion protein according to the third aspect, the recombinant cell according to the seventh aspect, and/or the pharmaceutical composition according to the ninth aspect, or treating the patient in need thereof using the drug box kit according to the fifteenth aspect.

The autoimmune disease, inflammatory disease, and/or allergic disease are as previously described.

The administration is as previously described.

In the present disclosure, the effective amount refers to a dose exhibiting an effect in diagnosing, preventing, and/or treating the autoimmune disease, inflammatory disease, and/or allergic disease. It is known to those skilled in the art that the dose is dependent on a variety of factors including formulation, route of administration, patient age, weight, gender, pathology, diet, time of administration, rate of excretion, and sensitivity of reaction.

A seventeenth aspect of the present disclosure provides a combination therapy for treating an autoimmune disease, an inflammatory disease, and/or an allergic disease, comprising administering to a patient in need thereof an effective amount of a first therapeutic agent and a second therapeutic agent; wherein the first therapeutic agent is the recombinant binding protein according to the first aspect, the recombinant binding protein according to the second aspect, the fusion protein according to the third aspect, the recombinant cell according to the seventh aspect, and/or the pharmaceutical composition according to the ninth aspect.

In some embodiments of the present disclosure, the second therapeutic agent comprises other recombinant binding proteins, anti-tumor antibodies, or pharmaceutical compositions comprising the same.

In some embodiments of the present disclosure, the second therapeutic agent is one or more selected from a hormone preparation, a small molecule targeted preparation, a proteasome inhibitor, an imaging agent, a diagnostic agent, a chemotherapeutic agent, an oncolytic drug, a cytotoxic agent, a cytokine, an activator of co-stimulatory molecules, and an inhibitor of inhibitory molecules.

An eighteenth aspect of the present disclosure provides a composition for diagnosing, preventing, and/or treating an autoimmune disease, an inflammatory disease, and/or an allergic disease, comprising the recombinant binding protein according to the first aspect, the recombinant binding protein according to the second aspect, the fusion protein according to the third aspect, the recombinant cell according to the seventh aspect, and/or the pharmaceutical composition according to the ninth aspect.

The autoimmune disease, inflammatory disease, and/or allergic disease are as previously described.

A nineteenth aspect of the present disclosure provides a kit, comprising the recombinant binding protein according to the first aspect, the recombinant binding protein according to the second aspect, the fusion protein according to the third aspect, the recombinant cell according to the seventh aspect, or the pharmaceutical composition according to the ninth aspect.

In some embodiments of the present disclosure, the kit further comprises an administration device for administering the recombinant binding protein according to the first aspect, the recombinant binding protein according to the second aspect, the fusion protein according to the third aspect, the recombinant cell according to the seventh aspect, or the pharmaceutical composition according to the ninth aspect.

A twentieth aspect of the present disclosure provides a method for determining TSLP expression, comprising determining TSLP expression using the recombinant binding protein according to the first aspect, the recombinant binding protein according to the second aspect, the fusion protein according to the third aspect, the recombinant cell according to the seventh aspect, or the pharmaceutical composition according to the ninth aspect.

A twenty-first aspect of the present disclosure provides a method for detecting or diagnosing an autoimmune disease, an inflammatory disease, and/or an allergic disease, comprising detecting TSLP expression using the recombinant binding protein according to the first aspect, the recombinant binding protein according to the second aspect, the fusion protein according to the third aspect, the recombinant cell according to the seventh aspect, or the pharmaceutical composition according to the ninth aspect.

A twenty-second aspect of the present disclosure provides a recombinant binding protein targeting TSLP, comprising at least one ankyrin repeat domain that specifically binds to human TSLP; the recombinant binding protein is capable of specifically binding to human TSLP, thereby blocking the formation of a complex among TSLP, TSLPR, and hIL-7Rα;
wherein the ankyrin repeat domain binds at least to one or more amino acid residues selected from D18, E20, K21, L27, S28, and S43 in the amino acid sequence as shown in SEQ ID NO: 17.

In some embodiments of the present disclosure, the ankyrin repeat domain binds at least to D18, E20, K21, L27, S28, and S43 in the amino acid sequence as shown in SEQ ID NO: 17.

The recombinant binding protein of the present disclosure has excellent binding activity to TSLP, effectively blocking the interaction between TSLP and a receptor thereof, for example, blocking the formation of a complex between TSLP and the receptor TSLPR/IL-7Rα, and can inhibit pro-inflammatory signaling that promotes type II immune response, thereby effectively controlling TSLP-related diseases such as allergic diseases.

### BRIEF DESCRIPTION OF THE DRAWINGS

FIG. 1 is a schematic diagram showing the results of specific binding of the screened 9 recombinant proteins to hTSLP by ELISA.

FIG. 2 is a schematic diagram showing the results of binding of hIL-7Rα-mFc to hTSLP/TSLPR-Fc complex by ELISA.

FIG. 3 is a schematic diagram showing the blocking effect of DARPin Fc recombinant protein on the interaction between hIL-7Rα-mFc and hTSLP/TSLPR-Fc complex by competitive ELISA.

FIG. 4 is a schematic diagram showing the results of the binding ability of 1A1-Fc fusion protein to hTSLP protein identified by ELISA.

FIG. 5 shows the blocking curve of 1A1-Fc on the interaction between hIL-7Rα-mFc and hTSLP/TSLPR-Fc complex.

FIG. 6 shows the blocking curve of 3H10-Fc on the interaction between hIL-7Rα-mFc and hTSLP/TSLPR-Fc complex.

FIG. 7 is a schematic diagram showing the results of the binding ability of 3H10-his recombinant protein to hTSLP-Fc protein identified by ELISA.

FIG. 8 shows the blocking curve of 3H10-his on the interaction between hIL-7Rα-mFc and hTSLP/TSLPR-Fc complex.

FIG. 9 shows the blocking curve of 3H10-his on the interaction between mouse IL-7Rα-his and hTSLP/hTSLPR-Fc complex.

FIG. 10 shows the fitting curve and IC50 value for the inhibition of the proliferation-promoting effect of hTSLP on Ba/F3-TSLPR/IL-7Rα cells by 3H10-his recombinant protein and Tezepelumab.

FIG. 11 shows the inhibition curve and IC50 value for the inhibition of hTSLP-induced reporter gene expression in BaF3-hTSLPR-hIL-7R-STAT5-Luc cells by 3H10-his recombinant protein and Tezepelumab.

FIG. 12 is a schematic diagram showing the inhibitory effect of 3H10-his recombinant protein on TSLP-stimulated CCL17 secretion by PBMCs.

### DETAILED DESCRIPTION OF THE PREFERRED EMBODIMENT

In the present disclosure, the term “tag” refers to an amino acid sequence linked to a polypeptide or protein that can be used for purification, detection, or targeting of the polypeptide or protein. The tag is conventional in the art, and may be, for example, a His tag.

### Example 1: DARPin Protein Panning Against hTSLP

hTSLP recombinant protein (purchased from ACROBiosystems, Cat. No. TSP-H52Ha) was coated onto a plate at 1 ug/well and incubated at 4° C. overnight. The next day, the plate was blocked with 1% skim milk at room temperature for 2 hours, added with 100 μL of phage (4×10E11 pfu/mL, DARPin phage display library), and the mixture was reacted at room temperature (20 to 25° C.) for 1 hour. Then, the plate was washed 10 times with PBST (PBS containing 0.05% (v/v) Tween 20) to remove unbound phages. Finally, the phage specifically binding to hTSLP was dissociated with triethylamine (100 mM), and infected into E. coli TG1 in logarithmic growth phase to produce and purify a phage for the next round of screening. The same screening process was repeated for 3 rounds. As a result, positive clones were enriched, and DARPins specifically binding to the hTSLP recombinant protein were obtained from the phage display library.

### Example 2: Screening of Specific Single Positive Clones Using Enzyme-Linked Immunosorbent Assay (ELISA) of Phage

After 3 rounds of panning as described in Example 1, the blank E. coli TG1 was infected with the obtained hTSLP binding positive phage and plated. Then, 96 single colonies were selected and cultured separately to produce and purify a phage as sample phage. hTSLP recombinant protein was coated onto a plate and incubated at 4° C. overnight. The obtained sample phage (the control group was a blank phage) was added, and the mixture was reacted at room temperature for 1 hour. After washing, the secondary antibody, mouse anti-M13 tag antibody (purchased from Beijing Sino Biological, 11973-MM05T-H) was added, and the mixture was reacted at room temperature for 1 hour. After washing, TMB substrate solution was added, and the absorbance was read at 450 nm. When the OD value of the sample well was more than 3 times of the OD value of the control well, the sample well was determined as a positive clone well. Bacteria from the positive clone well were transferred to LB liquid medium containing 100 μg/mL ampicillin and cultured for plasmid extraction and sequencing. Finally, A total of 9 different DARPin molecules were obtained, namely 1A11, 1A1, 1B9, 3H7, 2H4, 3B2, 2B12, 2C5, and 2G5, respectively.

### Example 3: Expression and Purification of Recombinant DARPin Fc Fusion Protein in CHO Cells

The coding sequences of the 9 DARPin molecules obtained by sequencing in Example 2 were subcloned into the expression vector pCDNA3.1+, and the correctly sequenced recombinant plasmid was transformed into the host bacteria Trans5α Chemically Competent Cell (TransGen, Cat. No. CD201-01), which were coated onto a plate of LB solid medium containing 100 μg/mL ampicillin and incubated at 37° C. overnight. Single colonies were selected for inoculation and incubated overnight. The next day, the bacterial solution was collected by centrifugation. The plasmid was extracted according to the HiPure Plasmid EF Mini Kit (Magen, Cat. No. P1112-03), and transfected into CHO cells (Thermo Fisher, A29127) to express the target protein. On the eleventh day, the supernatant was collected by centrifugation, and the fusion protein was purified by a Protein A affinity chromatography column. Finally, the antibody protein with a purity up to 90% or more was obtained.

### Example 4: Detection of Specific Binding of Candidate DARPin to Human TSLP Protein

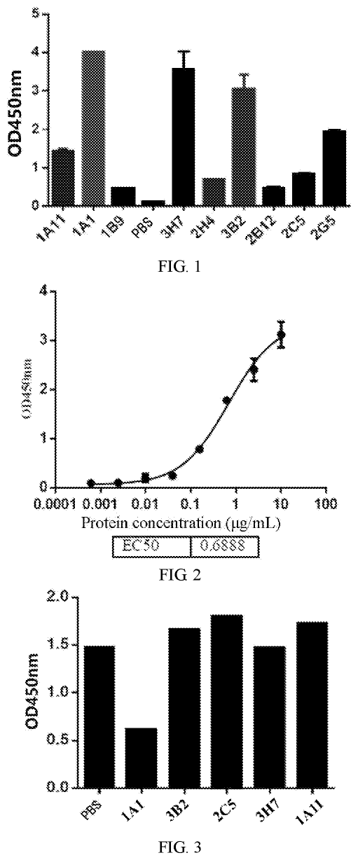

hTSLP recombinant protein (purchased from ACROBiosystems, Cat. No. TSP-H52Ha) was coated onto a plate at 0.1 μg/well and incubated at 4° C. overnight. Subsequently, the plate was washed and added with 100 ng/well of DARPin Fc recombinant protein obtained in Example 3 (the control group was DARPin recombinant protein not binding to hTSLP protein), and the mixture was reacted at room temperature for 1 hour. After washing, goat anti-human Fc tag horseradish peroxidase-labeled antibody (purchased from Abcam, ab98624) was added, and the mixture was reacted at room temperature for 1 hour. After washing, substrate solution was added, and the absorbance was read at 450 nm. When the ratio of the OD value of hTSLP protein to the OD value of the blank control was 4 or more, the candidate DARPin Fc recombinant protein was determined to be capable of specifically binding to the hTSLP protein. The results showed that all 9 DARPin Fc recombinant proteins screened can specifically bind to hTSLP. The specific results are shown in FIG. 1.

### Example 5: Identification of Binding of hIL-7Rα-mFc to hTSLP/TSLPR-Fc Complex by ELISA Assay

TSLPR-Fc recombinant protein (purchased from ACROBiosystems, Cat. No. TSR-H525a) was coated onto a plate at 0.1 μg/well and incubated at 4° C. overnight. Subsequently, the plate was washed and added with 0.1 μg/well of hTSLP, and the mixture was reacted at room temperature for 1 hour. After washing, 100 μL of hIL-7Rα-mFc (purchased from ACROBiosystems, Cat. No. ILA-H5258) was added at an initial concentration of 10 μg/mL with a serial dilution ratio of 1:4, and the mixture was reacted at room temperature for 1 hour. After washing, goat anti-mouse Fc tag horseradish peroxidase-labeled antibody (purchased from Abcam, ab97040) was added, and the mixture was reacted at room temperature for 1 hour. After washing, substrate solution was added, and the absorbance was read at 450 nm. As a result, the EC50 for the interaction between hIL-7Rα-mFc and hTSLP/TSLPR-Fc complex was 0.6888 μg/mL, as shown in FIG. 2.

### Example 6: Identification of the Blocking Effect of DARPin Fc Recombinant Protein on the Interaction Between hIL-7Rα-mFc and hTSLP/TSLPR-Fc Complex by Competitive ELISA Assay

TSLPR-Fc fusion protein was coated onto a plate at a concentration of 0.5 μg/mL and a volume of 100 μL per well, and incubated at 4° C. overnight. Subsequently, the plate was washed and added with 0.1 μg/well of hTSLP, and the mixture was reacted at room temperature for 1 hour. Subsequently, the plate was washed and added with 100 ng/well of DARPin Fc recombinant protein obtained in Example 3 and 50 ng/well of hIL-7Rα-mFc, and the mixture was reacted at room temperature for 1 hour. Then, goat anti-mouse Fc tag horseradish peroxidase-labeled antibody (purchased from Abcam, ab97040) was added, and the mixture was reacted at room temperature for 1 hour. Then, substrate solution was added, and the absorbance was read at 450 nm. When the ratio of the OD value of the sample to the OD value of the control was less than 0.8, the DARPin Fc recombinant protein was considered to have a blocking effect. As a result, the 1A1-Fc recombinant protein (amino acid sequence is SEQ ID NO: 3) exhibited a blocking effect on the interaction between hIL-7Rα-mFc and hTSLP/TSLPR-Fc complex, as shown in FIG. 3.

The amino acid sequence of 1A1 is as shown below:

(SEQ ID NO: 1)
GSDLGKKLLEAARAGRDDEVRILMANGADVNAGDASGYTPLHLAAYNGH
LEIVEVLLKHGADVNAEDLLGMTPLHLAAPFGHLEIVEVLLKHGADVNA
KNRNNGKTPLHLAAFVGHLEIVEVLLKNGADVNAQDKFGKTAFDISIDN
GNEDLAEILQKLNG

The nucleotide sequence of 1A1 is as shown below:

(SEQ ID NO: 2)
ggcagcgatctgggcaaaaaactgctggaagccgcccgcgccggccgcg
atgatgaagttcgcatcctgatggccaacggtgcggatgttaacgcggg
tgatgcttcgggttatacccctctgcatctggccgcctataatgggcat
ctggaaatcgttgaggttctgctgaaacatggcgcagatgttaatgccg
aggatctgctgggtatgacgccactgcatctggcggccccttttggcca
cctcgaaatagttgaggttcttttgaagcatggcgccgacgtaaacgcc
aagaatcggaataatggtaagactccgttgcacctggccgcatttgttg
ggcacctggagatcgtggaggttctcctcaaaaatggcgccgatgtcaa
tgcacaagataaattcggcaaaaccgccttcgatatcagcatcgataat
ggcaatgaagatctggccgaaatcctgcagaaactgaatggcca

The amino acid sequence of 1A1-Fc recombinant protein is as shown below:

(SEQ ID NO: 3)
GSDLGKKLLEAARAGRDDEVRILMANGADVNAGDASGYTPLHLAAYNGH
LEIVEVLLKHGADVNAEDLLGMTPLHLAAPFGHLEIVEVLLKHGADVNA
KNRNNGKTPLHLAAFVGHLEIVEVLLKNGADVNAQDKFGKTAFDISIDN
GNEDLAEILQKLNGEPKSCDKTHTCPPCPAPELLGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K

The amino acid sequence of Fc is as shown below:

(SEQ ID NO: 4)
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

### Example 7: Preparation of Monoclonal Antibody Targeting TSLP from AstraZeneca/Amgen

Anti-TSLP monoclonal antibody from AstraZeneca/Amgen, referring to the sequence of Tezepelumab in Patent US20180296669A1, has the specific amino acid sequence as shown below:

Heavy chain:
(SEQ ID NO: 5)
QMQLVESGGGVVQPGRSLRLSCAASGFTFRTYGMHWVRQAPGKGLEWVA
VIWYDGSNKHYADSVKGRFTITRDNSKNTLNLQMNSLRAEDTAVYYCAR
APQWELVHEAFDIWGQGTMVTVSS
Light chain:
(SEQ ID NO: 6)
SYVLTQPPSVSVAPGQTARITCGGNNLGSKSVHWYQQKPGQAPVLVVYD
DSDRPSWIPERFSGSNSGNTATLTISRGEAGDEADYYCQVWDSSSDHVV
FGGGTKLTVL

The antibody variable region gene sequence was synthesized (Shanghai LogenBio Co., Ltd.), cloned into the expression vector pCDNA3.1+, and transfected into CHO cells to express Tezepelumab.

### Example 8: Identification of Binding Ability of 1A1-Fc Fusion Protein to hTSLP Protein (ELISA)

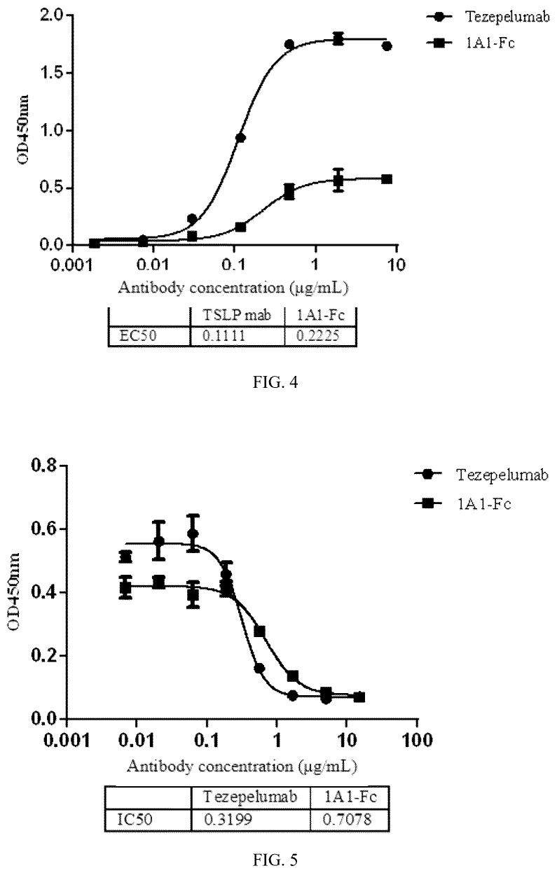

hTSLP recombinant protein was coated onto a plate at 0.05 μg/well and incubated at 4° C. overnight. Subsequently, the plate was washed and respectively added with a serial dilution series of 1A1-Fc fusion protein and Tezepelumab, with an initial concentration of 30 μg/mL, a volume of 100 μL, and a dilution ratio of 1:3, and the mixture was reacted at room temperature for 1 hour. After washing, goat anti-human Fc tag horseradish peroxidase-labeled antibody (purchased from Abcam, ab98624) was added, and the mixture was reacted at room temperature for 1 hour. After washing, substrate solution was added, and the absorbance was read at 450 nm. Data processing and graphical analysis were performed using the software Graphpad Prism 6.0. The binding curves and EC50 values for 1A1-Fc fusion protein and Tezepelumab to hTSLP were obtained by four-parameter fitting. As a result, the EC50 for the binding of 1A1-Fc fusion protein to hTSLP was 0.2225 μg/mL, while the EC50 for the binding of Tezepelumab to hTSLP was 0.1111 μg/mL, as shown in FIG. 4.

### Example 9: Identification of Binding Ability of 1A1-Fc Fusion Protein to hTSLP Protein (Gator)

1A1-Fc fusion protein or Tezepelumab was diluted to a concentration of 100 nM in KD buffer, and then immobilized onto anti-hIgG Fc probes. The association program of Gator Bio was then run to measure the binding rate constant for hTSLP protein diluted in KD buffer (200 nM, 100 nM, 50 nM, 25 nM, and 0 nM) binding to 1A1-Fc or Tezepelumab. Subsequently, the disassociation program was run to measure the dissociation constant for the binding of hTSLP protein to 1A1-Fc or Tezepelumab. The specific results are shown in Table 1:

TABLE 1
Dissociation constant for the binding of
hTSLP protein to 1A1-Fc or Tezepelumab

| | koff (1/s) | kon (1/Ms) | KD (M) |
|---|---|---|---|
| 1A1-Fc | 0.0116 | 8.72E+05 | 1.33E−08 |
| Tezepelumab | 0.000146 | 1.62E+06 | 8.96E−11 |

### Example 10: Blocking Curve of 1A1-Fc on the Interaction Between hIL-7Rα-mFc And hTSLP/TSLPR-Fc Complex

TSLPR-Fc fusion protein was coated onto a plate at a concentration of 0.5 μg/mL and a volume of 100 μL per well, and incubated at 4° C. overnight. Subsequently, the plate was washed and added with 0.25 μg/mL of hTSLP and hIL-7Rα-mFc as well as a serial dilution series of 1A1-Fc fusion protein and Tezepelumab (initial concentration of 15 μg/mL, 1:3 dilution) per well, and the mixture was reacted at room temperature for 1 hour. Then, goat anti-mouse Fc tag horseradish peroxidase-labeled antibody (1:5000) was added, and the mixture was reacted at room temperature for 1 hour. Then, TMD substrate solution was added, and the absorbance was read at 450 nm. Data processing and graphical analysis were performed using the software Graphpad Prism 6.0. The blocking curves and IC50 values for 1A1-Fc fusion protein and Tezepelumab on the interaction between hIL-7Rα-mFc and hTSLP/TSLPR-Fc complex were obtained by four-parameter fitting. As a result, the IC50 for 1A1-Fc fusion protein on the interaction between hIL-7Rα-mFc and hTSLP/TSLPR-Fc complex was 0.7078 μg/mL, while the IC50 for Tezepelumab was 0.3199 μg/mL, as shown in FIG. 5.

### Example 11: Affinity Maturation of 1A1 Clones

Multiple rounds of amplification were performed on the target gene fragment of 1A1 by error-prone PCR, followed by digestion, and ligation into pComb3× (Wuhan Miaoling Biotechnology Co., Ltd., P0862). Electroporation was performed on TG1 competent cells to construct a random mutation library of 1A1. The cells were inoculated into 2YT medium, incubated to an OD600 of approximately 0.6, and infected with M13KO7 (NEB, N0315S) for 30 minutes to amplify the 1A1 mutant phage display library. During the panning process, by decreasing the coating concentration of hTSLP antigen and increasing the number of PBST washing steps, the 1A1 mutant G7W1, which can bind to hTSLP with high affinity, was obtained through multiple rounds of panning, and the amino acid sequence of G7W1 is as shown in SEQ ID NO: 7. Subsequently, the above process was repeated with G7W1 as a template, resulting in obtaining 3H10 clone binding to hTSLP with high affinity finally, and the amino acid sequence of the 3H10 clone is as shown in SEQ ID NO: 8. The 3H10 gene sequence was cloned into the expression vector pCDNA3.1+, subject to transfect CHO cells to express the target protein, and the affinity of 3H10 for hTSLP was detected by Gator. The specific results are shown in Table 2:

TABLE 2
Affinity of 1A1, G7W1, and 3H10 to hTSLP

| | koff (1/s) | kon (1/Ms) | KD (M) |
|---|---|---|---|
| 1A1-Fc | 0.0118 | 8.62E+05 | 1.37E−08 |
| G7W1-Fc | 0.000365 | 6.25E+05 | 5.82E−10 |
| 3H10-Fc | 0.000124 | 5.68E+05 | 1.51E−10 |

The amino acid sequence of G7W1 is as shown below:

(SEQ ID NO: 7)
GSDLGKKLLEAAWAGRDDEVRILMANGADVNAGDASGDTPLHLAAVVGH
LEIVEVLLKHGADVNAEDLLGMTPLHLAAPFGHLEIVEVLLKHGADVNA
AARNNGKTPLHLAAFVGHLEIVEVLLKNGADVNAQDKFGKTAFDISIDN
GNEDLAEILQKLNG

The amino acid sequence of 3H10 is as shown below:

(SEQ ID NO: 8)
GSHMDLGKKLLEAAWAGRDDEVRILMANGADVNAGDASGDTPLHLAAFS
GHLEIVEVLLKHGADVNAEDLLGMTPLHLAAPFGHLEIVEVLLKHGADV
NAAARNNGKTPLHLAAFVGHLEIVEVLLKNGADVNAQDKFGKTAFDISI
DNGNEDLAEILQKLNG

### Example 12: Blocking Curve of 3H10-Fc on the Interaction Between hIL-7Rα-mFc and hTSLP/TSLPR-Fc Complex

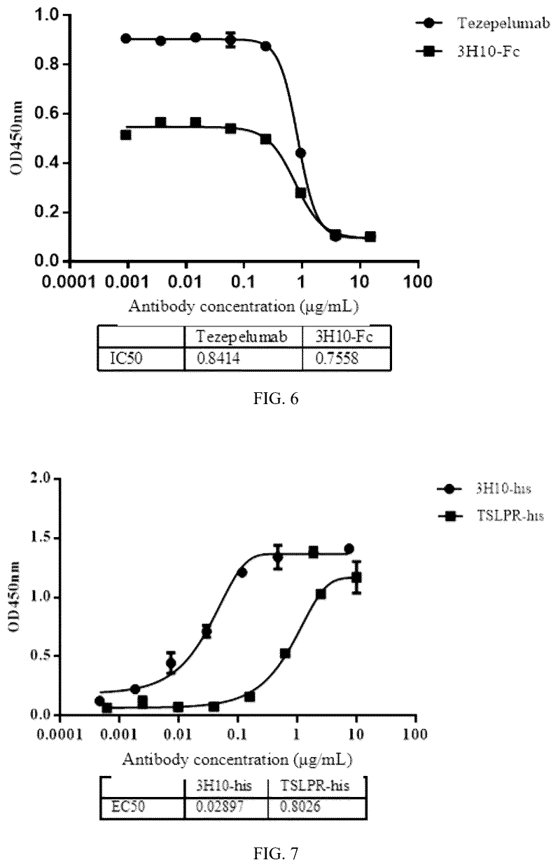

TSLPR-Fc fusion protein was coated onto a plate at a concentration of 0.5 μg/mL and a volume of 100 μL per well, and incubated at 4° C. overnight. Subsequently, the plate was washed and added with 0.25 μg/mL of hTSLP and hIL-7Rα-mFc as well as a serial dilution series of 3H10-Fc fusion protein and Tezepelumab (initial concentration of 15 μg/mL, 1:3 dilution) per well, and the mixture was reacted at room temperature for 1 hour. Then, goat anti-mouse Fc tag horseradish peroxidase-labeled antibody (1:5000) was added, and the mixture was reacted at room temperature for 1 hour. Then, TMD substrate solution was added, and the absorbance was read at 450 nm. Data processing and graphical analysis were performed using the software Graphpad Prism 6.0. The blocking curves and IC50 values for 3H10-Fc fusion protein and Tezepelumab on the interaction between hIL-7Rα-mFc and hTSLP/TSLPR-Fc complex were obtained by four-parameter fitting. As a result, the IC50 for 3H10-Fc fusion protein on the interaction between hIL-7Rα-mFc and hTSLP/TSLPR-Fc complex was 0.7558 μg/mL, while the IC50 for Tezepelumab was 0.8414 μg/mL, as shown in FIG. 6.

### Example 13: Prokaryotic Expression of 3H10

The target gene sequence of 3H10 (SEQ ID NO: 9) was synthesized according to the E. coli codon (AtaGenix Laboratories (Wuhan)), and subcloned into the expression vector pet28b. Subsequently, the plasmid was transformed into E. coli Rosetta competent cells, and the cells were coated onto a kanamycin-resistant LB plate, and incubated at 37° C. overnight. The next day, single colonies were picked and inoculated into 2 mL of LB medium (containing kanamycin), and incubated at 37° C. and 220 rpm overnight. On the third day, the culture was transferred to 200 mL of LB medium (containing kanamycin) at a 1% (v/v) inoculum, incubated to an OD600 of 0.6, and IPTG was added to induce expression at 30° C. and 220 rpm overnight. Then, the bacteria were harvested by centrifugation, resuspended in PBS buffer, disrupted by sonication (200 W, working for 5 seconds with an interval of 5 seconds, operating on ice) until clarified. The mixture was centrifuged (12000*g, 5 minutes) to collect the supernatant, which was then filtered, and 3H10-his was purified by a nickel affinity chromatography column. Finally, the recombinant protein with a purity of up to 90% or more was obtained.

The nucleotide sequence of 3H10 is as shown below:

(SEQ ID NO: 9)
ggcagccatatggatctgggcaaaaaactgctggaagcagcctgggcag
gtcgtgatgatgaagtgcgtattctgatggccaatggtgcagatgtgaa
tgccggtgacgccagcggtgacaccccgttacatctggcagcctttagc
ggccatctggaaattgtggaagttctgctgaaacatggtgccgatgtga
atgcggaagatctgctgggcatgaccccgctgcatctggcagcaccgtt
tggtcatctggaaatcgttgaagtgctgctgaaacacggtgccgatgtt
aatgccgccgcccgtaataatggtaaaaccccgctgcacctggccgcat
ttgttggtcatctggagattgtggaagtgctgttaaaaaatggcgccga
tgtgaacgcacaggataaatttggcaaaaccgcatttgatattagtatt
gataacggtaacgaggatctggcagaaattctgcagaaactgaatggt

The nucleotide sequence of G7W1 is as shown below:

(SEQ ID NO: 30)
ggcagcgatctgggcaaaaaactgctggaagccgcctgggccggccgcg
atgatgaagttcgcatcctgatggccaacggtgcggatgttaacgcggg
tgatgcttcgggtgatacccctctgcatctggccgccgttgttgggcat
ctggaaatcgttgaggttctgctgaaacatggcgcagatgttaatgccg
aggatctgctgggtatgacgccactgcatctggcggccccttttggcca
cctcgaaatagttgaggttcttttgaagcatggcgccgacgtaaacgcc
gctgctcggaataatggtaagactccgttgcacctggccgcatttgttg
ggcacctggagatcgtggaggttctcctcaaaaatggcgccgatgtcaa
tgcacaagataaattcggcaaaaccgccttcgatatcagcatcgataat
ggcaatgaagatctggccgaaatcctgcagaaactgaat

### Example 14: Identification of Binding Ability of 3H10-his Recombinant Protein to hTSLP-Fc Protein (ELISA)

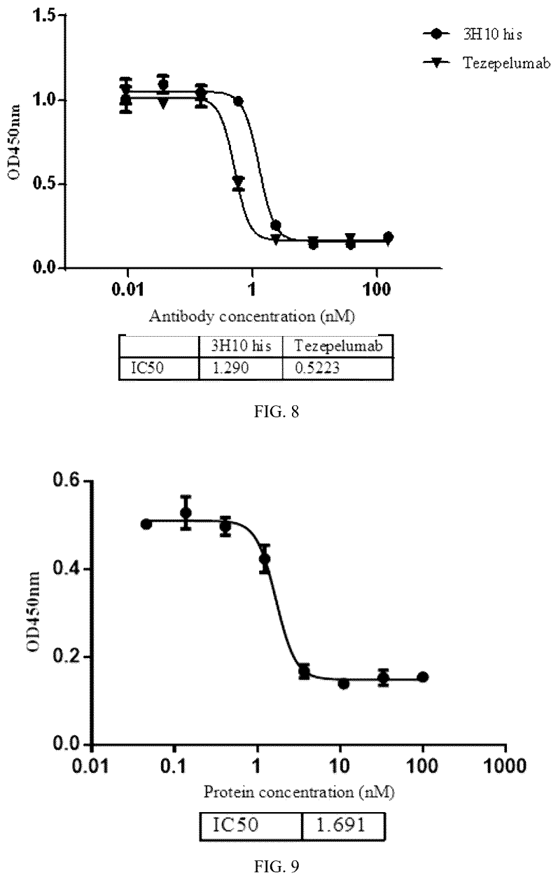

hTSLP-mFc recombinant protein (ACROBiosystems, Cat. No. TSP-H5255) was coated onto a plate at 50 ng/well and incubated at 4° C. overnight. Subsequently, the plate was washed and respectively added with a serial dilution series of 3H10-his recombinant protein and TSLPR-his (Novoprotein Scientific Inc., Cat. No. CW75), with an initial concentration of 10 μg/mL, a volume of 100 μL, and a dilution ratio of 1:4, and the mixture was reacted at room temperature for 1 hour. After washing, mouse anti-His tag horseradish peroxidase-labeled antibody (1:5000) (purchased from Beijing Sino Biological, 105327-MM02T-H) was added, and the mixture was reacted at room temperature for 1 hour. After washing, TMD substrate solution was added, and the absorbance was read at 450 nm. Data processing and graphical analysis were performed using the software Graphpad Prism 6.0. The binding curves and EC50 values for 3H10-his recombinant protein and TSLPR-his to hTSLP-mFc were obtained by four-parameter fitting. As a result, the EC50 of 3H10-his recombinant protein to hTSLP-Fc protein was 0.02897 μg/mL, while the EC50 of TSLPR-his was 0.8026 μg/mL, as shown in FIG. 7.

### Example 15: Blocking Curve of 3H10-his on the Interaction Between hIL-7Rα-mFc and hTSLP/TSLPR-Fc Complex

TSLPR-Fc fusion protein was coated onto a plate at a concentration of 0.5 μg/mL and a volume of 100 μL per well, and incubated at 4° C. overnight. Subsequently, the plate was washed and added with 0.25 μg/mL of hTSLP and hIL-7Rα-mFc as well as a serial dilution series of 3H10-his fusion protein and Tezepelumab (initial concentration of 150 nM, 1:4 dilution) per well, and the mixture was reacted at room temperature for 1 hour. Then, goat anti-mouse Fc tag horseradish peroxidase-labeled antibody (1:5000) was added, and the mixture was reacted at room temperature for 1 hour. Then, TMD substrate solution was added, and the absorbance was read at 450 nm. Data processing and graphical analysis were performed using the software Graphpad Prism 6.0. The blocking curves and IC50 values for 3H10-his fusion protein and Tezepelumab on the interaction between hIL-7Rα-mFc and hTSLP/TSLPR-Fc complex were obtained by four-parameter fitting. As a result, the IC50 for 3H10-his recombinant protein on the interaction between hIL-7Rα-mFc and hTSLP/TSLPR-Fc complex was 1.290 nM, while the IC50 for Tezepelumab was 0.5223 nM, as shown in FIG. 8.

### Example 16: Blocking Curve of 3H10-his on the Interaction Between Mouse IL-7Rα-his and hTSLP/hTSLPR-Fc Complex

Mouse IL-7Rα-his recombinant protein (purchased from Beijing Sino Biological, 50090-M08H) was coated onto a plate at a concentration of 1 μg/mL and a volume of 100 μL per well, and incubated at 4° C. overnight. Subsequently, the plate was washed and added with 0.5 μg/mL of hTSLP and 0.5 μg/mL of hTSLPR-Fc as well as a serial dilution series of 3H10-his fusion protein (initial concentration of 100 nM, 1:3 dilution) per well, and the mixture was reacted at room temperature for 1 hour. Then, goat anti-human Fc tag horseradish peroxidase-labeled antibody (1:10000) was added, and the mixture was reacted at room temperature for 1 hour. Then, TMD substrate solution was added, and the absorbance was read at 450 nm. Data processing and graphical analysis were performed using the software Graphpad Prism 6.0. The blocking curve and IC50 value for 3H10-his fusion protein on the interaction between mIL-7Rα-his and hTSLP/TSLPR-Fc complex were obtained by four-parameter fitting. As a result, the IC50 was 1.691 nM, as shown in FIG. 9.

### Example 17: Inhibition of the Proliferation-Promoting Effect of TSLP on Ba/F3-TSLPR/IL-7Rα Cells by 3H10-his Recombinant Protein

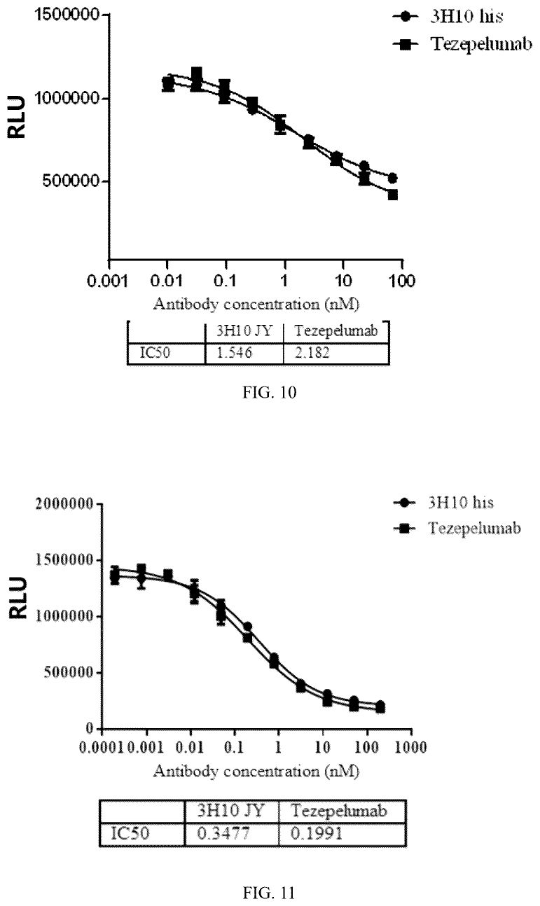

hTSLP can effectively promote the proliferation of Ba/F3-TSLPR/IL-7Rα cells (purchased from Cobioer), by which the inhibitory effect of monoclonal antibody targeting TSLP on TSLP during the proliferation of Ba/F3-TSLPR/IL-7Rα cells can be effectively evaluated. Before the experiment, Ba/F3-TSLPR/IL-7Rα cells were cultured in complete medium without hTSLP and starved for 24 hours. On the day of the experiment, cells were centrifuged (500*g, 5 minutes) and resuspended in GIBCO R1640 complete medium (containing 10% (v/v) FBS and 1% (v/v) PS (dual antibiotics: penicillin and streptomycin)), counted, and plated into a 96-well flat-bottom cell culture plate at 5000 cells per well (25 μL). hTSLP, pre-mixed with 3H10 or Tezepelumab in an equal volume for 30 minutes, was then added to medium in each well at a final concentration of 0.2 ng/mL. 3H10-his fusion protein and Tezepelumab were in a serial dilution series, starting at 66.6 nM and diluted 1:4, with a final volume of 100 μL. After 48 hours of incubation in a cell culture incubator, 50 μL of CTG reagent was added to each well. The plate was incubated in the dark for 5 minutes, and the fluorescence signal was read using a microplate reader (TECAN Spark). Data processing and graphical analysis were performed using the software Graphpad Prism 6.0. The blocking curves and IC50 values for the inhibition of the proliferation-promoting effect of hTSLP on Ba/F3-TSLPR/IL-7Rα cells by 3H10-his recombinant protein and Tezepelumab were obtained by four-parameter fitting. As a result, the IC50 for 3H10-his recombinant protein was 1.546 nM, while the IC50 for Tezepelumab was 2.182 nM, as shown in FIG. 10 (3H10JY is another batch of 3H10-his).

### Example 18: Inhibition of hTSLP-Induced Reporter Gene Expression in BaF3-hTSLPR-hIL-7R-STAT5-Luc Cells by 3H10-his Recombinant Protein

BaF3-hTSLPR-hIL-7R-STAT5-Luc reporter cells were harvested from a cell culture flask. Then, 50 μL of 1640 medium containing 50000 cells was inoculated onto a 96-well cell culture plate, with 1640 medium containing 10% (v/v) FBS. Meanwhile, 50 μL of hTSLP-his (ACROBiosystems, TSP-H52Ha) (dissolved in 1640 medium containing 10% FBS at a final concentration of 0.25 ng/ml) was separately mixed with 50 μL of serially diluted (starting at 200 nM, serially diluted 5-fold in 1640 medium containing 10% FBS) 3H10 recombinant protein and Tezepelumab, and the mixture was incubated at room temperature for 30 minutes. Then, a mixture of anti-TSLP antibody/TSLP-his was added to the 96-well cell culture plate at 50 μL/well, and incubated at 37° C. in a CO₂ incubator for 4.5 hours. 100 μL of One-lite reagent (Nanjing Vazyme, DD120303) was added, and the plate was shaken for 5 minutes to mix thoroughly. The chemiluminescence signal was read using a microplate reader. The data of the luminescence signal were analyzed using Graphpad Prism software and the IC50 values were obtained, As a result, the IC50 for recombinant protein was 0.3477 nM, while the IC50 for Tezepelumab was 0.1991 nM, as shown in FIG. 11 (3H10JY is another batch of 3H10-his).

### Example 19: Inhibitory Effect of 3H10-his Recombinant Protein on TSLP-Stimulated CCL17 Secretion from PBMCs

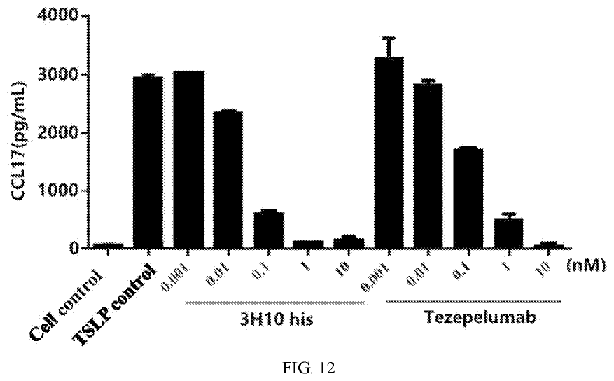

After hTSLP binds to TSLPR on dendritic cells, the formed complex further binds to IL-7Rα, leading to the activation of dendritic cells, which is manifested by the secretion of the chemokine CCL17. By detecting the expression of CCL17, the inhibitory effect of 3H10-his recombinant protein on hTSLP can be effectively evaluated. PBMCs (purchased from Milestone Biotechnologies Co., Ltd.) were resuspended in R1640 medium containing 10% serum to a concentration of 10⁶ cells/mL, and plated into a 96-well flat-bottom cell culture plate at 10⁵ cells/100 μL per well. Both 3H10-his recombinant protein and the control antibody Tezepelumab were serially diluted 10-fold starting at a final concentration of 20 nM (a total of four concentration gradients). The diluted 3H10-his or Tezepelumab was premixed with hTSLP-his at a final concentration of 2 ng/mL in an equal volume for 30 minutes, added to cells and mixed to a total volume of 200 μL/well, and the mixture was incubated at 37° C. with 5% CO₂ for 24 hours. The supernatant was then collected and CCL17 was detected using Human CCL17/TARC Antibody (purchased from R&D Systems, MAB364), Human CCL17/TARC Biotinylated Antibody (purchased from R&D Systems, BAF364), and Recombinant Human CCL17/TARC Protein (purchased from R&D Systems, 364-DN-025). The OD450 absorbance was read using a microplate reader (TECAN Spark). Data processing and graphical analysis were performed using the software Graphpad Prism 6.0. As a result, both 3H10-his fusion protein and the control antibody Tezepelumab exhibited an effective inhibitory effect on hTSLP, as shown in FIG. 12.

### Example 20: Identification of Antigen Epitope of 3H10-his Binding to hTSLP

Through homology modeling of 3H10 and docking predictions of 3H10 and hTSLP complexes, some of the amino acids of hTSLP were identified as potentially playing an important role in binding to 3H10-his. Subsequently, based on the principle of alanine scanning, the mutated target gene fragments of hTSLP were sequentially synthesized and cloned into the expression vector pCDNA3.1+. Then, the vector was transfected into CHO cells to express hTSLP mutants, which were analyzed for their affinity to 3H10-biotin by Gator. The specific TSLP mutations are as follows: hTSLP-D18A, hTSLP-E20A, hTSLP-K21A, hTSLP-K23A, hTSLP-L27A, hTSLP-S28A, hTSLP-K32A, hTSLP-S43A, hTSLP-R55A, hTSLP-R127A, hTSLP-N130A, hTSLP-R131A, hTSLP-L133A, and hTSLP-K135A. The difference between the binding of 3H10-biotin protein to wild-type hTSLP and the binding of 3H10-biotin protein to hTSLP mutants is shown in Table 3. As a result, amino acids D18, E20, K21, L27, S28, and S43 play an important role in the binding of 3H10 to hTSLP recombinant protein.

The amino acid sequence of hTSLP is as shown below:

(SEQ ID NO: 17)
YDFTNCDFEKIKAAYLSTISKDLITYMSGTKSTEFNNTVSCSNRPHCLT
EIQSLTFNPTAGCASLAKEMFAMKTKAALAIWCPGYSETQINATQAMKK
ARKAKVTTNKCLEQVSQLQGLWRRFNRPLLKQQ

TABLE 3
Decreased fold in binding affinity of each
TSLP mutant compared to wild-type TSLP

| | TSLP mutants | Decreased fold |
|---|---|---|
| 1 | D18A | 7.9 |
| 2 | E20A | 6.1 |
| 3 | K21A | 11.9 |
| 4 | K23A | 2.4 |
| 5 | L27A | 9.3 |
| 6 | S28A | 5.1 |
| 7 | K32A | 1.2 |
| 8 | S43A | 5 |
| 9 | R55A | 0.5 |
| 10 | R127A | 1.8 |
| 11 | N130A | 2.2 |
| 12 | R131A | 2.1 |
| 13 | L133A | 2.05 |
| 14 | K135A | 1.7 |
| 15 | WT | — |

Although specific embodiments of the present disclosure have been described above, it should be understood by those skilled in the art that these are merely illustrative, and various changes or modifications can be made to these embodiments without departing from the principles and spirit of the present disclosure. Accordingly, the scope of the present disclosure is defined by the appended claims.

1. A recombinant binding protein targeting TSLP, comprising at least one ankyrin repeat domain that specifically binds to TSLP;
the ankyrin repeat domain comprises three tandem binding domains; the amino acid sequence of the binding domain is as shown in SEQ ID NO: 22; or,
the recombinant binding protein targeting TSLP comprises at least one ankyrin repeat domain that specifically binds to TSLP; the ankyrin repeat domain comprises three tandem binding domains, and each binding domain comprises four binding active sites; wherein the three binding domains are a first binding domain, a second binding domain, and a third binding domain in tandem; the first binding domain, the second binding domain, the third binding domain, and a fourth binding domain each comprise a first binding active site, a second binding active site, a third binding active site, and a fourth binding active site in tandem; the amino acid sequence of the first binding active site of the first binding domain is as shown in SEQ ID NO: 10, the amino acid residue of the second binding active site is Y or D, the amino acid sequence of the third binding active site is YN, VV, or FS, and the amino acid residue of the fourth binding active site is H; the amino acid sequence of the first binding active site of the second binding domain is as shown in SEQ ID NO: 11, the amino acid residue of the second binding active site is M, the amino acid sequence of the third binding active site is PF, and the amino acid residue of the fourth binding active site is H; the amino acid sequence of the first binding active site of the third binding domain is as shown in SEQ ID NO: 12 or SEQ ID NO: 13, the amino acid residue of the second binding active site is K, the amino acid sequence of the third binding active site is FV, and the amino acid residue of the fourth binding active site is N.

2. The recombinant binding protein according to claim 1, wherein the amino acid sequence of the binding domain is as shown in any one of SEQ ID NOs: 23 to 28.

3. (canceled)

4. The recombinant binding protein according to claim 1, wherein at least one ankyrin repeat domain of the recombinant binding protein has the amino acid sequence as shown in SEQ ID NO: 1, 7, or 8; or, the recombinant binding protein comprises two, three, or four ankyrin repeat domains.

5. A fusion protein targeting TSLP, comprising the recombinant binding protein according to claim 1 and a structurally stable protein; the structurally stable protein is used for prolonging the in vivo plasma half-life of the recombinant binding protein.

6-8. (canceled)

9. A recombinant cell comprising the recombinant binding protein according to claim 1 or a fusion protein;
wherein the fusion protein comprises the recombinant binding protein and a structurally stable protein; the structurally stable protein is used for prolonging the in vivo plasma half-life of the recombinant binding protein.

10. (canceled)

11. A pharmaceutical composition comprising the recombinant binding protein according to claim 1 or a fusion protein, and a pharmaceutically acceptable carrier;
wherein the fusion protein comprises the recombinant binding protein and a structurally stable protein; the structurally stable protein is used for prolonging the in vivo plasma half-life of the recombinant binding protein.

12. (canceled)

13. A drug box kit comprising a drug box A and a drug box B;
the drug box A comprises the recombinant binding protein according to claim 1 or a fusion protein; the drug box B comprises other therapeutic agents;
wherein the fusion protein comprises the recombinant binding protein and a structurally stable protein; the structurally stable protein is used for prolonging the in vivo plasma half-life of the recombinant binding protein.

14. A method for preventing or treating an autoimmune disease, an inflammatory disease, or an allergic disease, comprising administering to a patient in need thereof an effective amount of the recombinant binding protein according to claim 1 or a fusion protein;
wherein the fusion protein comprises the recombinant binding protein and a structurally stable protein; the structurally stable protein is used for prolonging the in vivo plasma half-life of the recombinant binding protein.

15. A recombinant binding protein targeting TSLP, comprising at least one ankyrin repeat domain that specifically binds to human TSLP; the recombinant binding protein is capable of specifically binding to human TSLP, thereby blocking the formation of a complex among TSLP, TSLPR, and hIL-7Rα;
wherein the ankyrin repeat domain binds at least to one or more amino acid residues selected from D18, E20, K21, L27, S28, and S43 in the amino acid sequence as shown in SEQ ID NO: 17.

16. The recombinant binding protein according to claim 2, wherein the three tandem binding domains are a first binding domain, a second binding domain, and a third binding domain, respectively; wherein the amino acid sequence of the first binding domain is as shown in SEQ ID NO: 23, SEQ ID NO: 26, or SEQ ID NO: 27; the amino acid sequence of the second binding domain is as shown in SEQ ID NO: 24; the amino acid sequence of the third binding domain is as shown in SEQ ID NO: 25 or SEQ ID NO: 28.

17. The recombinant binding protein according to claim 16, wherein the ankyrin repeat domain further comprises an N-terminal capping region and a C-terminal capping region; the amino acid sequence of the N-terminal capping region is as shown in SEQ ID NO: 29, the amino acid sequence of the C-terminal capping region is as shown in SEQ ID NO: 21.

18. The recombinant binding protein according to claim 17, wherein the amino acid sequence of the N-terminal capping region is as shown in any one of SEQ ID NOs: 18 to 20.

19. The recombinant binding protein targeting TSLP according to claim 1, wherein the amino acid sequence of the first binding active site of the first binding domain is as shown in SEQ ID NO: 10, the amino acid residue of the second binding active site is Y, the amino acid sequence of the third binding active site is YN, and the amino acid residue of the fourth binding active site is H; the amino acid sequence of the first binding active site of the second binding domain is as shown in SEQ ID NO: 11, the amino acid residue of the second binding active site is M, the amino acid sequence of the third binding active site is PF, and the amino acid residue of the fourth binding active site is H; the amino acid sequence of the first binding active site of the third binding domain is as shown in SEQ ID NO: 12, the amino acid residue of the second binding active site is K, the amino acid sequence of the third binding active site is FV, and the amino acid residue of the fourth binding active site is N; or,
the amino acid sequence of the first binding active site of the first binding domain is as shown in SEQ ID NO: 10, the amino acid residue of the second binding active site is D, the amino acid sequence of the third binding active site is VV, and the amino acid residue of the fourth binding active site is H; the amino acid sequence of the first binding active site of the second binding domain is as shown in SEQ ID NO: 11, the amino acid residue of the second binding active site is M, the amino acid sequence of the third binding active site is PF, and the amino acid residue of the fourth binding active site is H; the amino acid sequence of the first binding active site of the third binding domain is as shown in SEQ ID NO: 13, the amino acid residue of the second binding active site is K, the amino acid sequence of the third binding active site is FV, and the amino acid residue of the fourth binding active site is N; or
the amino acid sequence of the first binding active site of the first binding domain is as shown in SEQ ID NO: 10, the amino acid residue of the second binding active site is D, the amino acid sequence of the third binding active site is FS, and the amino acid residue of the fourth binding active site is H; the amino acid sequence of the first binding active site of the second binding domain is as shown in SEQ ID NO: 11, the amino acid residue of the second binding active site is M, the amino acid sequence of the third binding active site is PF, and the amino acid residue of the fourth binding active site is H; the amino acid sequence of the first binding active site of the third binding domain is as shown in SEQ ID NO: 13, the amino acid residue of the second binding active site is K, the amino acid sequence of the third binding active site is FV, and the amino acid residue of the fourth binding active site is N.

20. The recombinant binding protein targeting TSLP according to claim 19, wherein the binding domain further comprises a framework site; the framework site comprises a first framework site, a second framework site, a third framework site, and a fourth framework site sequentially; the binding activity site and the framework site are arranged with intervals; wherein the amino acid residue of the first framework site is G, the amino acid sequence of the second framework site is as shown in SEQ ID NO: 14, the amino acid sequence of the third framework site is as shown in SEQ ID NO: 15, and the amino acid sequence of the fourth framework site is shown as SEQ ID NO: 16.

21. The recombinant binding protein targeting TSLP according to claim 20, wherein the N-terminus and C-terminus of the ankyrin repeat domain further comprise a capping sequence; wherein the amino acid sequence of the capping sequence at the N-terminus is as shown in SEQ ID NO: 18, SEQ ID NO: 19, or SEQ ID NO: 20; the amino acid sequence of the capping sequence at the C-terminus is as shown in SEQ ID NO: 21.

22. A drug box kit comprising a drug box A and a drug box B;
the drug box A comprises the recombinant cell according to claim 9; the drug box B comprises other therapeutic agents.

23. A drug box kit comprising a drug box A and a drug box B;
the drug box A comprises the pharmaceutical composition according to claim 11; the drug box B comprises other therapeutic agents.

24. A method for preventing or treating an autoimmune disease, an inflammatory disease, or an allergic disease, comprising administering to a patient in need thereof an effective amount of the recombinant cell according to claim 9.

25. A method for preventing or treating an autoimmune disease, an inflammatory disease, or an allergic disease, comprising administering to a patient in need thereof an effective amount of the pharmaceutical composition according to claim 11.

26. A method for preventing or treating an autoimmune disease, an inflammatory disease, or an allergic disease, comprising administering to a patient in need thereof an effective amount of the drug box kit according to claim 13.

27. The method according to claim 14, wherein the autoimmune disease is selected from rheumatoid arthritis and multiple sclerosis; the inflammatory disease is selected from ulcerative colitis, eosinophilic esophagitis, chronic obstructive pulmonary disease, and psoriasis; the allergic disease is selected from atopic dermatitis, allergic rhinitis, allergic conjunctivitis, asthma, and allergic sinusitis.
